(19) United States
(12) Patent Application Publication
(10) Pub. No.: US 2006/0178737 A1
(43) Pub. Date: Aug. 10, 2006

(54) Coated medical apparatus

(76) Inventor: Leo T. Furcht, Golden Valley, MN (US)

Correspondence Address: KONDZELLA AND CYR, PONDVIEW PLAZA, SUITE 114, 5850 OPUS PARKWAY, MINNETONKA, MN, 55343, US

(21) Appl. No.: 11/332,592

(22) Filed: Jan. 12, 2006

Related U.S. Application Data
(60) Provisional application No. 60/643,507, filed on Jan. 12, 2005.

(51) Int. Cl. A61F 2/24; A61F 2/28; A61F 2/82

(52) U.S. Cl. 623/1.46; 623/2.42; 623/23.51

(57) ABSTRACT

Medical apparatus for medical treatment of a patient are disclosed. The medical apparatus include a diamond coating on at least a portion of the surface of the body of the medical apparatus.

### CROSS-REFERENCE TO RELATED APPLICATIONS

The present application claims priority from a U.S. Provisional Patent Application having Ser. No. 60/643,507 filed Jan. 12, 2005 the disclosure of which is hereby incorporated by reference in its entirety.

### BACKGROUND OF THE INVENTION

### Field of the Invention

The present invention related to medical devices and, more particularly, to surfaces of medical devices.

### SUMMARY OF THE INVENTION

Apparatus and methods in accordance with the present inventions may resolve many of the needs and shortcomings in the art of medical apparatus that will be recognized by those skilled in the art upon review of the present disclosure.

The present inventions provide a medical apparatus having a diamond coating on at least a portion of the surface of the body forming the medical apparatus. The diamond coating can confer desired physical and structural characteristics to the surface of the medical apparatus which provide advantages as will be recognized by those skilled in the art. The medical apparatus in accordance with the present invention may include one or more layers of a diamond coating secured to at least a portion of a surface of a body of a medical apparatus.

Other features and advantages of the invention will become apparent from the following detailed description, and from the claims.

### BRIEF DESCRIPTION OF THE DRAWINGS

FIG. 1 illustrates a perspective view of an embodiment of an intravascular stent in accordance with the present invention;

FIG. 2 illustrates a perspective view of an embodiment of a prosthetic hip in accordance with the present invention;

FIG. 3 illustrates a cross-section of an embodiment of a prosthetic hip in accordance with the present invention implanted in a patient;

FIG. 4 illustrates a perspective view of an embodiment of a prosthetic knee joint in accordance with the present invention;

FIG. 5 illustrates a cross-section of an embodiment of a prosthetic knee join in accordance with the present invention implanted in a patient;

FIG. 6 illustrates a partial perspective view of an embodiment of a catheter for insertion into a lumen of a patient in accordance with the present invention;

FIG. 7 illustrates a transverse cross-section through the embodiment of a catheter illustrated in FIG. 6;

FIG. 8 illustrates perspective view of an embodiment of a prosthetic heart valve in accordance with the present invention;

FIG. 9 illustrates an embodiment of a medical electrical lead in accordance with the present invention; and

FIG. 10 illustrates an embodiment of a medical electrical lead in accordance with the present invention.

### DETAILED DESCRIPTION OF THE INVENTION

The figures generally illustrate embodiments of medical apparatus 10 including aspects of the present inventions. The particular exemplary embodiments of the occluding guidewires 10 illustrated in the figures have been chosen for ease of explanation and understanding of various aspects of the present inventions. These illustrated embodiments are not meant to limit the scope of coverage but instead to assist in understanding the context of the language used in this specification and the appended claims. Accordingly, many variations from the illustrated embodiments may be encompassed by the appended claims.

The present invention provides a novel surfacing material for use with medical devices. In one aspect, the present invention provides a diamond coating that is deposited on the surface or surfaces of a medical device. The material may be deposited using chemical vapor deposition (CVD) processes such as those disclosed in U.S. Pat. Pub. No. 2003/0131787 and U.S. Pat. No. 6,582,513 the disclosures of which are hereby incorporated by reference in their entirety. In other aspects, the present invention may provide a diamond coating that is embedded or secured to a surface or surfaces of a medical device. In one configuration the surface may be comprised solely of a diamond coating. In other aspects, the surface structure may comprise a composite of diamond coating as well as other materials. The other materials may include biologically active compounds. Some biologically active compounds may include, among others, drugs, antibodies, hormones, growth factors or inhibitors of cell growth. In one aspect, the composite material may be formed by copolymerization of diamond films or structures with other organics and non-organic molecules.

The diamond coated surface may be configured to abut tissue when implanted in a patient. In such a configuration, the diamond coated surface may be positioned on the apparatus to interface with a tissue of the patient. In such a configuration, the diamond coated surface may, for example, provide a surface that is less thrombogenic and has a greater biocompatibility. In another configuration, the diamond coated surfaces may coat one or more of the surfaces of the medical device that interface with one another, such as for example the ball and the socket portions of an artificial hip. In such a configuration the diamond coated surfaces may, for example, provide a surface that is both lubricious and durable.

FIG. 1 illustrates a perspective view of a medical apparatus 10 in the form of stent for implantation within the lumen of a patient. An exemplary medical apparatus 10 in the form of a stent is described in U.S. Pat. No. 6,461,380 the disclosure of which is hereby incorporated by reference. A stent 10 generally comprises a tubular body 14 configured to be enlarged from a contracted configuration to an expanded configuration to generally support a lumen in an open position. The body 14 is frequently composed of a stainless steel, nitinol™ or polymeric material. In accordance with the present invention, a diamond coating 12 or a diamond composite material 12 is secured to or embedded in at least a portion of the exterior surface of the medical apparatus 10. In one aspect, the stent 10 may include a uniform diamond coating 12 desposited over the entire surface of its body 14 using CVD. In other aspects, the stent 10 may include a diamond coating 12 over at least a portion of its body 14 that has been deposited by CVD. In still other aspects, the coating diamond coating 12 is mechanically embedded in or adhesively secured to at least a portion of the body 12. In still another aspect, the diamond coating 12 may be localized to surfaces of body 14 which are in contact with blood in situ.

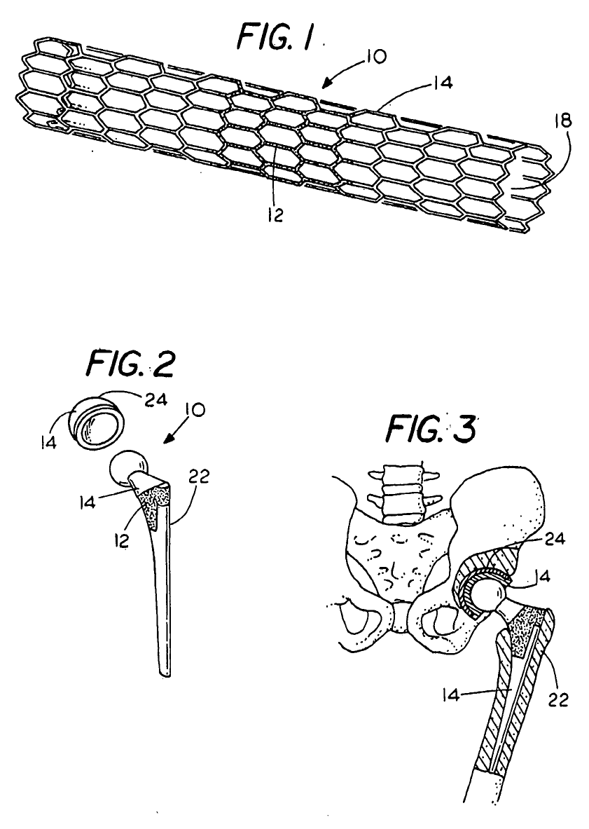

FIGS. 2 and 3 illustrate an embodiment of a medical apparatus 10 in the form of a prosthetic hip joint. An exemplary medical apparatus 10 with the body 14 in the form of a prosthetic hip joint is described in U.S. Pat. No. 6,726,725 the disclosure of which is hereby incorporated by reference. As illustrated, a prosthetic hip joint 10 generally includes a ball portion 22 configured to be secured in the femur and a socket portion 24 to be secured in the pelvis. The ball portion 22 includes an enlongated body 14 to be secured in the femur as is generally illustrated in FIG. 3. The socket portion 24 includes a cup shaped body 14 configured to be secured in the pelvis and to movably receive the ball of the ball portion 22. The body 14 of the ball portion 22 may include a diamond coating 12 deposited or embedded on at least a portion of its exterior surface. In one aspect, the diamond coating 12 is deposited using CVD. In one aspect, the diamond coating 12 is deposited, embedded or secured to a surface of at least a portion of the body 14 of the ball portion 22 and/or the socket portion 24 that interface with the bone or other tissues of the patient. In another aspect, the diamond coating 12 is deposited, embedded or otherwise secured to at least a portion of a surface of the body 14 of the ball portion 22 or the socket portion 24 that articulate with one another.

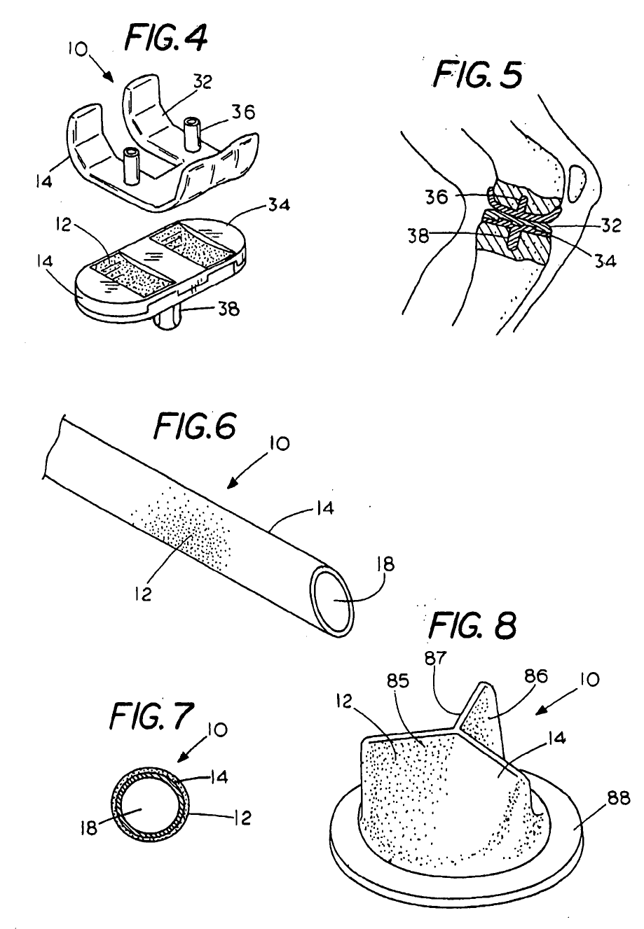

FIGS. 4 and 5 illustrate an embodiment of a medical apparatus 10 in the form of a prosthetic knee joint. An exemplary medical apparatus 10 with the body 14 in the form of a prosthetic knee joint is described in U.S. Pat. No. 6,726,725 the disclosure of which is hereby incorporated by reference. As illustrated, a prosthetic knee joint 10 generally includes a femoral portion 32 configured to be secured in the femur and a tibial portion 34 to be secured in the tibia. The femoral portion 32 may include one or more femoral anchors 36 to secure the femoral portion 32 within the femur of a patient as is generally illustrated in FIG. 5. The tibial portion 32 may include one or more tibial anchors 38 to secure the tibial portion 34 to the tibia of a patient as is also generally illustrated in FIG. 5. The bodies 14 of the femoral portion 32 and the tibial portion 36 are further configured to articulate with one another to emulate the function of a healthy knee. The body 14 of the femoral portion 32 and/or the tibial portion 34 may include a diamond coating 12 deposited or embedded on at least a portion of their exterior surface. In one aspect, the diamond coating may be deposited using CVD. In one aspect, the diamond coating 12 is deposited, embedded or secured to a surface of at least a portion of the body 14 of the femoral portion 32 and/or the tibial portion 34 that interface with the bone or other tissues of the patient. In another aspect, the diamond coating 12 is deposited, embedded or otherwise secured to at least a portion of a surface of the body 14 of the femoral portion 32 and/or the tibial portion 34 that articulate with one another.

FIGS. 6 and 7 illustrate an embodiment of a medical apparatus 10 in the form of a catheter. An exemplary medical apparatus 10 with the body 14 in the form of a catheter is described in U.S. Pat. No. 5,921,957 the disclosure of which is hereby incorporated by reference. A catheter 10 is generally configured as a tube to be inserted within the lumen of a patient for the introduction or removal of materials. Accordingly, a catheter 10 generally includes a body 14 defining a lumen 18. In accordance with the present invention, a diamond coating 12 is deposited, embedded or secured to a surface of at least a portion of the body 14 of a catheter 10. In one aspect, the diamond coating 12 may be deposited using CVD. As illustrated, the diamond coating 12 is secured to an exterior surface of the body 10 which will be the portion of the catheter 10 which has the most extensive contact with tissues of the patient. In other aspects, the diamond coating 12 may be deposited, secured or embedded in at least a portion of the interior surface of the body 14 within lumen 18.

FIG. 8 illustrates an embodiment of a medical apparatus 10 in the form of an artificial heart valve. An exemplary medical apparatus 10 with the body 14 in the form of an artificial heart valve is described in U.S. Pat. No. 6,761,736 the disclosure of which is hereby incorporated by reference. A heart valve 10 is generally configured to passively open and close due to differential pressure created by the beating of the heart. Heart valve 10 generally consists of a set of three leaflets 85, 86, 87 secure to a ring 88. The leaflets 85, 86, 87 are typically made from a flexible material 14 that permits their opening and closing with the beating of the heart. Ring 88 secures the leaflets 85, 86, 87 within the patient and may be constructed from the same or a distinct material 14. The material 14 of the Ring 88 and/or leaflets 85, 86, 87 may include a diamond coating 12 deposited or embedded on at least a portion of their exterior surface. In one aspect, the diamond coating may be deposited using CVD. In one aspect, the diamond coating 12 is deposited, embedded or secured to a surface of at least a portion of the material 14 of the Ring 88 and/or leaflets 85, 86, 87 that interface with the blood or other tissues of the patient. In another aspect, the diamond coating 12 is deposited, embedded or otherwise secured to at least a portion of a surface of the material 14 of the leaflets 85, 86, 87 that articulate with one another.

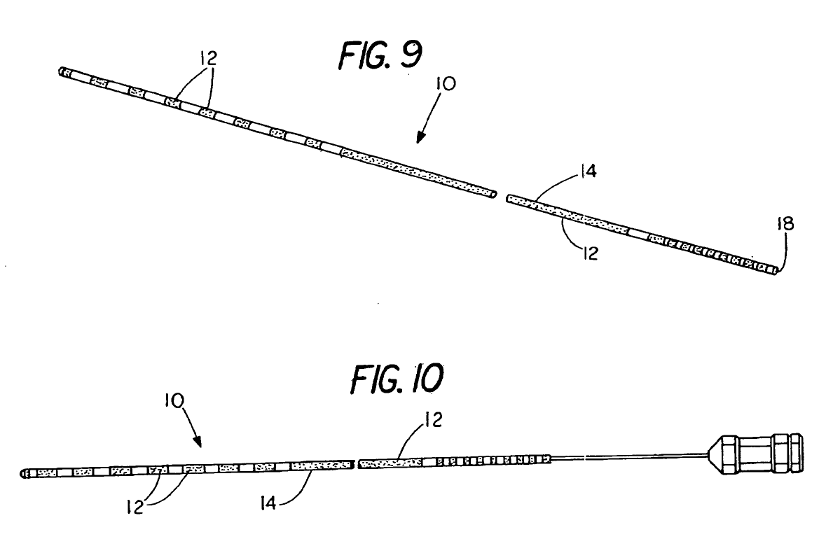

FIGS. 9 and 10 illustrate an embodiment of a medical apparatus 10 in the form of an implantable lead. An exemplary medical apparatus 10 with the body 14 in the form of an implantable lead is described in U.S. Pat. No. 6,216,045 the disclosure of which is hereby incorporated by reference. A lead 10 generally includes a connector end and an tissue stimulating/sensing end. A lead 10 may be configured for pacing, defibrillation, neurostimulation, cochlear implantation among other applications. A lead 10 generally includes an elongated body 14 comprising an insulating material surrounding one or more insulated conductors. Lead 10 may define a lumen 18. The conductors terminate on electrodes positioned at the connector end and the tissue stimulating/sensing end to relay an electrical stimulus between the patient and a medical device. The body 14 may include a diamond coating 12 deposited or embedded on at least a portion of its exterior surface. In one aspect, the diamond coating may be deposited using CVD. In one aspect, the diamond coating 12 is deposited, embedded or secured to a surface of at least a portion of the body 14 of the lead 10 that interfaces with tissues of the patient. In another aspect, the diamond coating 12 is deposited, embedded or otherwise secured to at least a portion of a surface of the body 14 of the lead 10 that articulates with the medical device to which it is connected.

In view of the exemplary embodiments, the present disclosure, and the disclosures incorporated by reference, those skilled in the art will appreciate the application of a diamond coatings 12 in conjunction with in a wide range of medical apparatus 10. One additional set of medical apparatus 10 are cardiovascular diagnostic devices, including venous blood pressure manometers, electrode recording catheters, electrode recording probes, flow-directed catheters, percutaneous catheters, steerable catheters, catheter cannulas, catheter guide wires, catheter introducers, catheter stylets, trocars, cardiovascular monitoring devices, catheter tip pressure transducers, ultrasonic transducers, and vessel occlusion transducers, among others. Another additional set of medical apparatus 10 are cardiovascular prosthetic devices, including vascular clips, vena cava clips, arterial embolization devices, cardiovascular intravascular filters, vascular graft prosthesis, implantable pacemaker pulse generators, pacemaker lead adaptors, cardiovascular permanent pacemaker electrodes, cardiovascular permanent temporary pacemaker electrodes, annuloplasty rings, carotid sinus nerve stimulators, replacement heart valves, prosthetic heart valve holder, and prosthetic heart valve sizers, among others. Another additional set of medical apparatus 10 are cardiovascular therapeutic devices such as embolectomy catheters, among others. Another set of medical apparatus 10 are prosthetic devices, including prosthetic devices made from gold-based alloys and precious metal alloys for clinical use. Another set of medical apparatus 10 are prosthetic devices, including ortho-implants, such as: bone cap; bone fixation cerclage; bone heterograft; intramedullary fixation rod; passive tendon prosthesis; polymethylmethacrylate (PMMA) bone cement; single/multiple component metallic bone fixation appliances and accessories; smooth or threaded metallic bone fixation fastener; resorbable calcium salt bone void filler device; spinal interlaminal fixation orthosis; spinal intervertebral body fixation orthosis; pedicle screw spinal system; ankle joint metal/composite semi-constrained cemented prosthesis; ankle joint metal/polymer semi-constrained cemented prosthesis; ankle joint metal/polymer non-constrained cemented prosthesis; elbow joint metal/polymer constrained cemented prosthesis; elbow joint metal/polymer semi-constrained cemented prosthesis; elbow joint radial (hemi-elbow) polymer prosthesis; elbow joint humeral (hemi-elbow) metallic uncemented prosthesis; finger joint metal/metal constrained uncemented prosthesis; finger joint metal/metal constrained cemented prosthesis; finger joint metal/polymer constrained cemented prosthesis; finger joint polymer constrained prosthesis; hip joint metal constrained cemented or uncemented prosthesis; hip joint metal/polymer constrained cemented or uncemented prosthesis; hip joint metal/metal semi-constrained, with a cemented acetabular component, prosthesis; hip joint metal/metal semi-constrained, with an uncemented acetabular component, prosthesis; hip joint metal/composite semi-constrained cemented prosthesis; hip joint metal/polymer semi-constrained cemented prosthesis; hip joint metal/ceramic/polymer semi-constrained cemented or nonporous uncemented prosthesis; hip joint metal/polymer/metal semi-constrained porous-coated uncemented prosthesis; hip joint femoral (hemi-hip) metallic cemented or uncemented prosthesis; hip joint (hemi-hip) acetabular metal cemented prosthesis; hip joint femoral (hemi-hip) trunnion-bearing metal/polyacetal cemented prosthesis; hip joint femoral (hemi-hip) metal/polymer cemented or uncemented prosthesis; hip joint femoral (hemi-hip) metallic resurfacing prosthesis; hip joint metal/polymer semi-constrained resurfacing cemented prosthesis; knee joint femorotibial metallic constrained cemented prosthesis; knee joint femorotibial metal/composite non-constrained cemented prosthesis; knee joint femorotibial metal/composite semi-constrained cemented prosthesis; knee joint femorotibial metal/polymer constrained cemented prosthesis; knee joint femorotibial metal/polymer non-constrained cemented prosthesis; knee joint femorotibial metal/polymer semi-constrained cemented prosthesis; knee joint femorotibial (uni-compartmental) metal/polymer porous-coated uncemented prosthesis; knee joint patellofemoral polymer/metal semi-constrained cemented prosthesis; knee joint patellofemorotibial polymer/metal/metal constrained cemented prosthesis; knee joint patellofemorotibial polymer/metal/polymer semi-constrained cemented prosthesis; Knee joint patellofemorotibial metal/polymer porous-coated uncemented prosthesis; knee joint femoral (hemi-knee) metallic uncemented prosthesis; knee joint patellar (hemi-knee) metallic resurfacing uncemented prosthesis; knee joint tibial (hemi-knee) metallic resurfacing uncemented prosthesis; shoulder joint metal/metal or metal/polymer constrained cemented prosthesis; shoulder joint metal/polymer non-constrained cemented prosthesis; shoulder joint metal/polymer semi-constrained cemented prosthesis; shoulder joint metal/polymer/metal nonconstrained or semi-constrained porous-coated uncemented prosthesis; shoulder joint glenoid (hemi-shoulder) metallic cemented prosthesis; shoulder joint humeral (hemi-shoulder) metallic uncemented prosthesis; Toe joint polymer constrained prosthesis; toe joint phalangeal (hemi-toe) polymer prosthesis; wrist joint carpal lunate polymer prosthesis; wrist joint carpal scaphoid polymer prosthesis; wrist joint carpal trapezium polymer prosthesis; wrist joint polymer constrained prosthesis; Wrist joint metal constrained cemented prosthesis; wrist joint metal/polymer semi-constrained cemented prosthesis; and wrist joint ulnar (hemi-wrist) polymer prosthesis, among others. Another set of medical apparatus 10 are orthopedic manual surgical instruments. Another set of medical apparatus 10 are obstetrical and gynecological surgical devices, including obstetric-gynecologic general manual instruments and obstetric-gynecologic specialized manual instrument. Another set of medical apparatus 10 are assisted reproductive devices. Another set of medical apparatus 10 are neurological surgical devices, including: dowel cutting instruments; manual cranial drills, burrs, trephines, and their accessories; powered compound cranial drills, burrs, trephines, and their accessories; cranial drill handpieces; microsurgical instruments; stereotaxic instruments; and neurosurgical suture needles, among others

Another set of medical apparatus 10 are neurological therapeutic devices, including cranial electrotherapy stimulators, implanted cerebellar stimulators, implanted diaphragmatic/phrenic nerve stimulators, implanted intracerebral/subcortical stimulators for pain relief, implanted spinal cord stimulators for bladder evacuation, implanted peripheral nerve stimulators for pain relief, implanted spinal cord stimulators for pain relief, artificial embolization devices. Another set of medical apparatus 10 are general hospital and personal use therapeutic devices, including intravascular catheters, intravascular needles, hypodermic single lumen needles, and percutaneous, implanted, long-term intravascular catheters, among others. Another set of medical apparatus 10 are general and plastic surgery devices, including manual or robotic surgical instruments for general use, laser surgical instruments for use in general and plastic surgery and in dermatology. Yet another set of medical apparatus 10 are auditory prosthetic devices, including hearing aids, cochlear implants, partial ossicular replacement prosthesis, and total ossicular replacement prosthesis, among others.

In addition, those skilled in the art will appreciate the applications for diamond structures constructed using CVD, as disclosed in U.S. Pat. Pub. No. 2003/0131787 and U.S. Pat. No. 6,582,513, in various molecular diagnostic devices, including MEMS/NEMS based, detection of molecules, proteins, hormones, chemicals, antibodies, virus, bacteria, DNA, RNA, cDNA, etc such as, for example, those disclosed in U.S. Pat. No. 6,303,288 the disclosure of which is hereby incorporated by reference as well as for use as nanoparticles for biological modulation such as, for example, those applications disclosed in U.S. Pub. No. 2003/0038303 the disclosure of which is hereby incorporated by reference.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

What is claimed is:

1. A medical apparatus, comprising a body having at least a portion of a surface of the body having a diamond coating.

2. A medical apparatus, as in claim 1, further comprising the diamond coating deposited on at least a portion of the surface of the body using chemical vapor deposition.

3. A medical apparatus, comprising a body having at least a portion of a surface of the body having a diamond coating wherein the medical apparatus is implantable.

4. A medical apparatus, as in claim 3, further comprising the diamond coating deposited on at least a portion of the surface of the body using chemical vapor deposition.

5. A medical apparatus, as in claim 3, further comprising the body defining an implantable lead.

6. A medical apparatus, as in claim 3, further comprising the body defining a artificial heart valve.

7. A medical apparatus, as in claim 3, further comprising the body defining an intravascular stent.

8. A medical apparatus, as in claim 3, further comprising the body defining a prosthetic hip joint.

9. A medical apparatus, as in claim 3, further comprising the body defining a catheter.

10. A medical apparatus, as in claim 3, further comprising the body defining a prosthetic hip joint.
